(19) United States
(12) Patent Application Publication
(10) Pub. No.: US 2023/0138069 A1
(43) Pub. Date: May 4, 2023

(54) SAFETY SYRINGE CAPABLE OF BEING USED FOR NEEDLE-FREE DOSING INJECTION

(71) Applicant: Chih-Hua HUNG, HSINCHU CITY (TW)

(72) Inventor: Chih-Hua HUNG, HSINCHU CITY (TW)

(21) Appl. No.: 17/684,675

(22) Filed: Mar. 2, 2022

(30) Foreign Application Priority Data
Nov. 1, 2021 (TW) 110140618

(51) Int. Cl. A61M 5/32; A61M 5/34; A61M 5/30

(52) U.S. Cl. CPC A61M 5/3213; A61M 5/343; A61M 5/3286; A61M 5/30; A61M 2005/3206

(57) ABSTRACT

A safety syringe includes a needle seat, an adapter disposed on the needle seat, and an injection barrel penetrated into the needle seat and the adapter. When an outer sleeve is located at a first position, a first annular rib of the outer sleeve is engaged with an elastic wing of the adapter, such that the needle seat protrudes from the front end of the outer sleeve. A push rod penetrated into the injection barrel is used to take in and expel liquid medicine. When the outer sleeve is located at a second position, the outer sleeve accommodates the needle seat and the adapter inside and pushes against the elastic wind of the adapter through a second annular rib, such that the outer sleeve is detachable from the injection barrel together with the needle seat and the adapter, The injection barrel can be used for needle-free dosing injection.

### BACKGROUND OF THE INVENTION

### 1. Field of the Invention

The present invention relates to a safety syringe and more particularly, to a safety syringe that can be used for needle-free dosing injection.

### 2. Description of the Related Art

As far as a conventional safety syringe is concerned, after injection, a push rod and a needle seat are engaged with each other by means of a special structural design, such that the push rod can be pulled into an injection barrel together with the needle seat and a needle, preventing medical personnel or medical waste handlers from being injured by the used needle accidentally. However, after use of the needle, since the needle seat and the needle can't be separated from each other, the medical personnel will throw away the needle seat and the needle together. Although the needle has been used, the structure of the injection barrel is usually still intact. This will cause waste of material and increase manufacturing costs.

### SUMMARY OF THE INVENTION

It is a primary objective of the present invention to provide a safety syringe, which can be used with a needle-free dosing connector for enhancing convenience of use and reducing waste of material.

To attain the above objective, the safety syringe of the present invention comprises a needle seat, an adapter, an injection barrel, and an outer sleeve. The front end of the needle seat is adapted for mounting a needle, and the rear end of the needle seat has a first shaft hole. The adapter has a second shaft hole through its front and rear ends. The second shaft hole of the adapter is sleeved on the rear end of the needle seat, and the second shaft hole of the adapter communicates coaxially with the first shaft hole of the needle seat, An elastic wing is provided at the outer surface of the adapter, The injection barrel has a barrel body and a shaft portion integrally connected with the front end of the barrel body and detachably penetrated into the first shaft hole of the needle seat and the second shaft hole of the adapter. The outer sleeve has a front opening and a rear opening, and the inner surface of the outer sleeve has a first annular rib adjacent to the front opening and a second annular rib adjacent to the rear opening. The outer sleeve is sleeved on the injection barrel and movable along the axial direction of the injection barrel.

When liquid medicine is drawn, the outer sleeve is pulled backwards to a first position where the first annular rib of the outer sleeve is engaged with the elastic wing of the adapter, such that the outer sleeve is sleeved on the barrel body of the injection barrel, and the needle seat and the needle are allowed to protrude from the front opening of the outer sleeve. At this time, the needle can enter a medicine bottle, and the liquid medicine is drawn into the injection barrel by using a push rod penetrated into the injection barrel. After drawing of the liquid medicine, there are two different ways to inject the liquid medicine. The first way is to push the push rod for performing injection of the liquid medicine through the needle. The second way is to push forwards the outer sleeve to a second position where the outer sleeve accommodates the needle seat and the adapter inside, Thereafter, the outer sleeve is pushed continuously to push against the elastic wind of the adapter through the second annular rib, such that the outer sleeve is detached from the injection barrel together with the needle seat, the needle, and the adapter. At this time, the shaft portion of the injection barrel is connected with a needle-free dosing connector, and then the push rod is pushed to perform injection of the liquid medicine.

It can be understood from the above illustration that the outer sleeve is pulled to the second position before the liquid medicine is drawn for preventing others from being injured by the needle. When the liquid medicine is drawn, the outer sleeve is pulled to the first position to expose the needle for facilitating drawing of the liquid medicine, After drawing of the liquid medicine, the liquid medicine is injected by using the needle or the needle-free dosing connector according to actual needs for enhancing convenience of use.

Preferably, the outer surface of the needle seat has an outer flange, The inner surface of the adapter has an inner flange interfered with the outer flange of the needle seat to prevent the needle seat from being separated from the adapter through the top end of the second shaft hole.

Preferably, the outer surface of the barrel body of the injection barrel has an elongated rib forced against the second annular rib of the outer sleeve when the outer sleeve is located at the first position for performing the positioning of the outer sleeve.

Preferably, the inner surface of the outer sleeve has a third annular rib adjacent to the second annular rib and located between the first annular rib and the second annular rib. When the outer sleeve is located at the second position, the elastic wing of the adapter is located between the second annular rib and the third annular rib for ensuring the needle to be received in the outer sleeve.

Preferably, the inner diameter of the second annular rib is greater than the inner diameter of the first annular rib and the inner diameter of the third annular rib, As such, the first annular rib or the third annular rib can be allowed to easily pass through the elastic wing of the adapter without applying too much force to the outer sleeve.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an assembled sectional plan view of a safety syringe of the present invention.

FIG. 2 is an exploded sectional plan view of the safety syringe of the present invention.

FIG. 3 is a bottom view of an adapter provided by the safety syringe of the present invention.

FIG. 4 is a sectional view taken along line 4-4 of FIG. 3,

FIG. 5 is similar to FIG. 1, showing that the outer sleeve is located at the second position.

FIG. 6 is similar to FIG. 5, showing that the outer sleeve is detached from the injection barrel together with the needle, the needle seat, and the adapter.

FIG. 7 is another assembled sectional plan view of the safety syringe of the present invention, showing that liquid medicine is drawn through the needle.

FIG. 8 is similar to FIG. 7, showing that liquid medicine is injected through the needle.

FIG. 9 is another assembled sectional plan view of the safety syringe of the present invention, showing that the outer sleeve is detached from the injection barrel together with the needle, the needle seat, and the adapter after performing drawing of the liquid medicine.

FIG. 10 is similar to FIG. 9, showing that the shaft portion of the injection barrel is connected with a needle-free dosing connector.

### DETAILED DESCRIPTION OF THE INVENTION

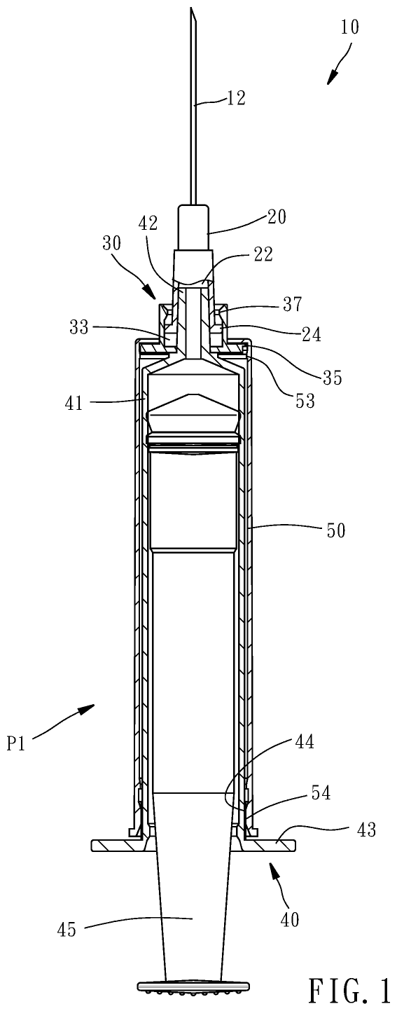

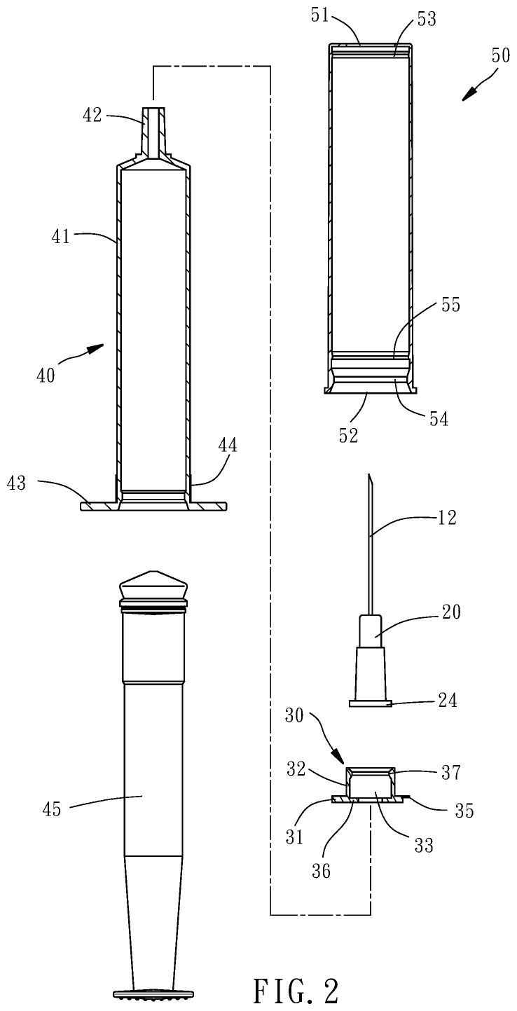

Referring to FIGS. 1 and 2, a safety syringe 10 of the present invention comprises a needle seat 20, an adapter 30. an injection barrel 40, a push rod 45, and an outer sleeve 50.

As shown in FIG. 2, the front end of the needle seat 20 is used for mounting with the rear end of a needle 12. The rear end of the needle seat 20 has a first shaft hole 22, and the outer surface of the rear end of the needle seat 20 has an outer flange 24.

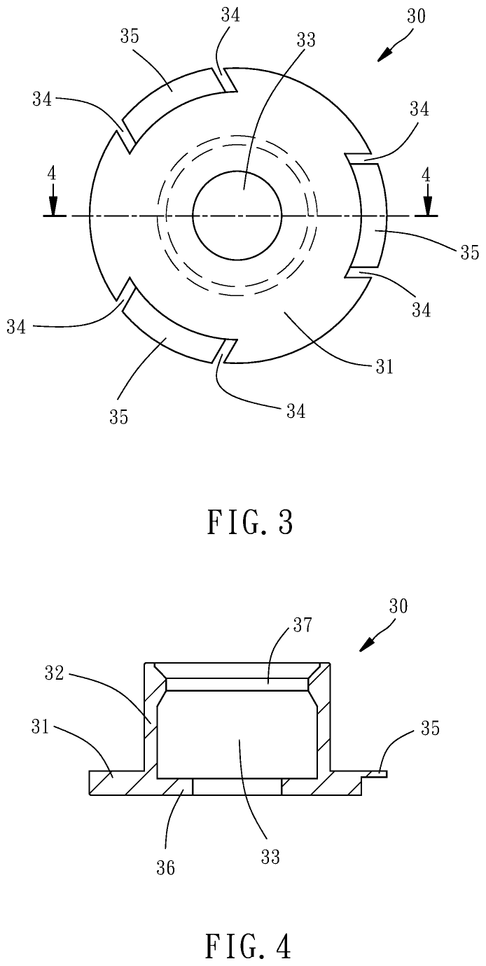

As shown in FIGS. 3 and 4, the adapter 30 has a lager annular portion 31, a small annular portion 32, and a second shaft hole 33 through the lager annular portion 31 and the small annular portion 32. Three pairs of notches 34 are provided at the outer surface of the large annular portion 31, An elastic wing 35 is formed between the two adjacent notches 34. The three elastic wings 35 are arranged in an equally-spaced manner relative to the second shaft hole 33. The inner surface of the large annular portion. 31 has a baffle 36 protruding along the radial direction of the second shaft hole 33. In addition, the inner surface of the small annular portion 32 has an inner flange 37 extending along the radially direction of the second shaft hole 33. As shown in FIGS. 1 and 2, the second shaft hole 33 of the adapter 30 is sleeved on the rear end of the needle seat 20, and the inner flange 37 is interfered with the outer flange 24 of the needle seat 20. By this way, the adapter 30 uses the baffle 36 to prevent the needle seat 20 from being separated from the adapter 30 through the rear end of the second shaft hole 33, and the adapter 30 uses the inner flange 37 to prevent the needle seat 20 from being separated from the adapter 30 through the front end of the second shaft hole 33.

As shown in FIG. 2, the injection barrel 40 has a barrel body 41, a shaft portion 42 connected with the front end of the barrel body 41, and a support portion 43 connected with the rear end of the barrel body 41. A plurality of elongated ribs 44 (the number is unlimited) are provided at the outer surface of the rear end of the barrel body 41 and arranged in an equally-spaced manner relative to the axial direction of the injection barrel 40. As shown in FIG. 1, the shaft portion 42 of the injection barrel 40 is penetrated into the first shaft hole 22 of the needle seat 20 and the second shaft hole 33 of the adapter 30 through the rear end of the adapter 30.

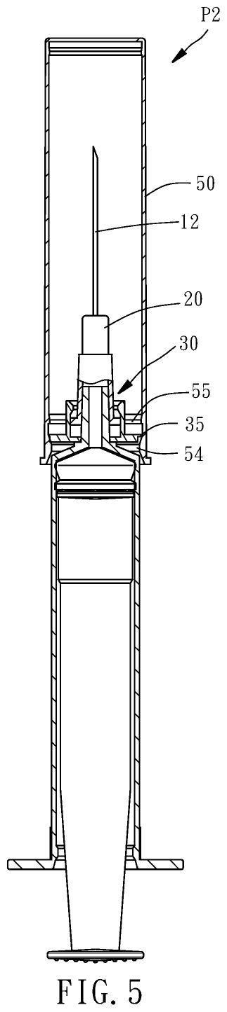

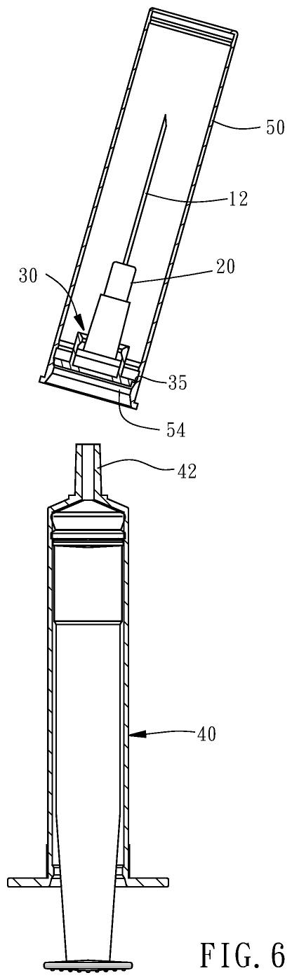

As shown in FIG. 2, the outer sleeve 50 has a front opening 51 and a rear opening 52. The outer sleeve 50 has a first annular rib 53 adjacent to the front opening 51 and a second annular rib 54 adjacent to the rear opening 52. As shown in FIGS. 1 and 5, the outer sleeve 50 is sleeved on the injection barrel 40 and movable along the axial direction of the injection barrel 40. When the outer sleeve 50 is moved backwards to a position where the first annular rib 53 is abutted with the elastic wings 35 of the adapter 30, the elastic wings 35 of the adapter 30 are deformed elastically for allowing the first annular rib 53 to pass through without being stopped. When the outer sleeve 50 is moved to a first position PI as shown in FIG. 1, the elastic wings 35 of the adapter 30 return to original status so as to be engaged with the first annular rib 53 of the outer sleeve 50, such that the outer sleeve 50 is sleeved on the barrel body 41 of the injection barrel 40, and the needle seat 20 and the needle 12 are allowed to protrude from the front opening 51 of the outer sleeve 50, and further, the second annular rib 54 of the outer sleeve 50 are forced against the elongated ribs 44 of the injection barrel 40 for performing the positioning of the outer sleeve 50. When the outer sleeve 50 is moved forwards to a position where the third annular rib 55 is abutted with the elastic wings 35 of the adapter 30, the elastic wings 35 of the adapter 30 are deformed elastically for allowing the third annular rib 55 to pass through without being stopped. When the outer sleeve 50 is moved forwards to a second position P2 as shown in FIG. 5, the outer sleeve 50 accommodates the needle seat 20, the needle 12, and the adapter 30 inside, and further, the elastic wings 35 of the adapter 30 are located between the second annular rib 54 and the third annular rib 55 for performing the positioning of the outer sleeve 50. In addition, under the condition that the outer sleeve 50 is located at the second position P2, when the outer sleeve 50 is pushed continuously to move forwards, as shown in FIG. 6, the outer sleeve 50 pushes against the elastic wings 35 of the adapter 30 through the second annular rib 54 because the inner diameter of the second annular rib 54 is greater than the inner diameter of the first annular rib 53 and the inner diameter of the third annular rib 55. As such, the outer sleeve 50 is detached from the injection barrel 40 together with the needle seat 20, the needle 12, and the adapter 30.

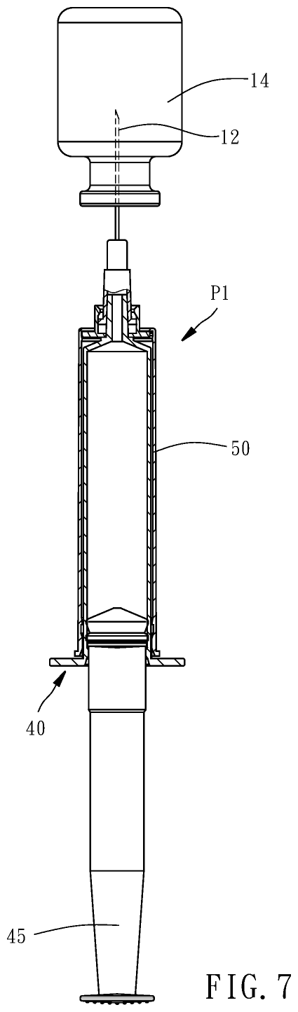

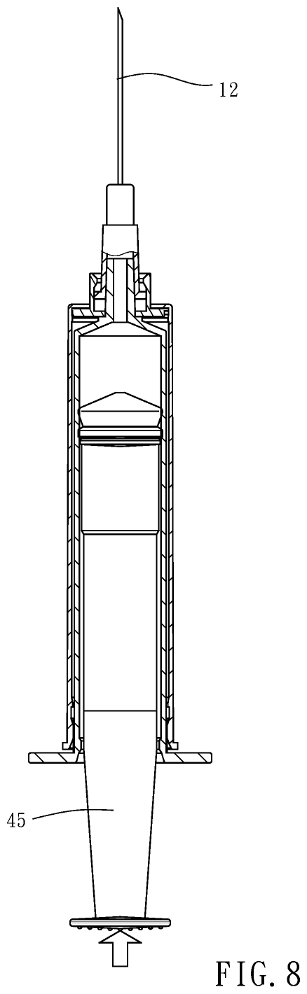

It can be seen from the above that before drawing liquid medicine into the injection barrel 40, a user pushes the outer sleeve 50 forwards to the second position P2 as shown in FIG. 5 for preventing others from being injured by the needle 12. When drawing the liquid medicine, the user pulls the outer sleeve 20 backwards to the first position P1 as shown in FIG. 7 to expose the needle 12 to the outside. At this time, the user inserts the needle 12 into a medicine bottle, and then pulls the push rod 45 to draw the liquid medicine into the injection barrel 40. After drawing of the liquid medicine, if it is unnecessary to inject the liquid medicine immediately, the user can push the outer sleeve 50 forwards to the second position P2 as shown in FIG. 5 for protecting the needle 12 from contamination; moreover, the user can draw the liquid medicine into several injection barrels 40 for subsequent use. When preparing to inject the liquid medicine, the user pulls the outer sleeve 50 backwards to the first position P1, and then pushes the push rod 54 to inject the liquid medicine through the needle 12, as shown in FIG. 8. Therefore, the safety syringe 10 of the present invention is quite convenient to use.

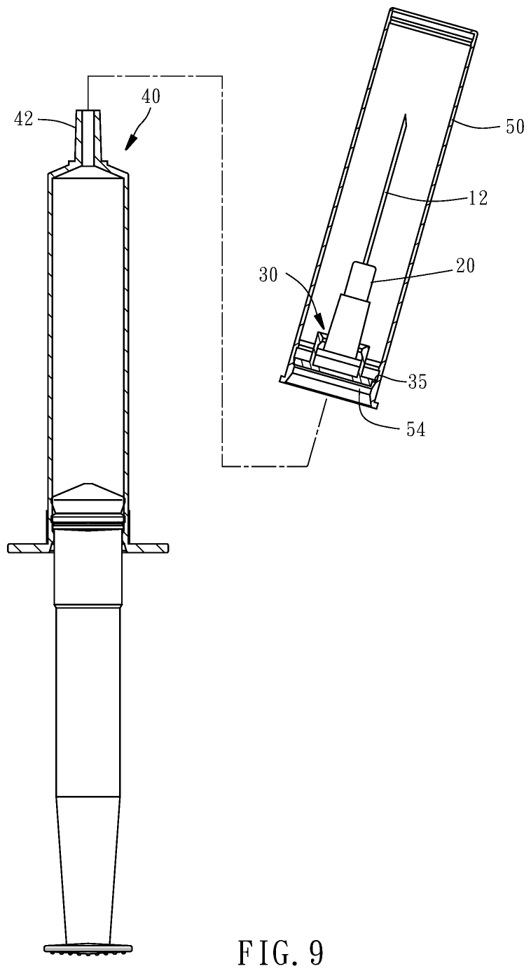

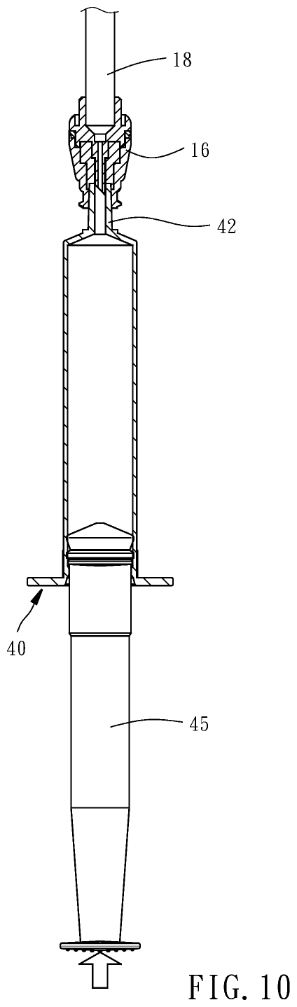

In addition to be used with the needle 12, the safety syringe 10 of the present invention can be used with a needle-free dosing connector 16, As shown in FIG. 9, after drawing of the liquid medicine, the user pushes the outer sleeve 50 forwards to the second position P2, and then pushes the outer sleeve 50 continuously, such that the outer sleeve 50 is pushed against the elastic wings 35 of the adapter 30 through the second annular rib 54. By this way, the outer sleeve 50 is detached from the injection barrel 40 together with the needle seat 20, the needle 12, and the adapter 30. At this time, the shaft portion 42 of the injection barrel 40 is connected with the needle-free dosing connector 16, as shown in FIG. 10, and then the push rod 45 is pushed to inject the liquid medicine into an infusion tube 18 through the needle-free dosing connector 16. Since the needle 12 is detached in advance, the situation that the infusion tube 18 is punctured accidentally by the needle 12 can be avoided.

As indicated above, when the liquid medicine is drawn, the outer sleeve 50 is pulled to the first position P1 to expose the needle 12 to the outside for facilitating drawing of the liquid medicine. After the liquid medicine is drawn, the liquid medicine can be injected by using the needle 12 or the needle-free dosing connector 16 according to actual needs for enhancing convenience of use. In other words, the safety syringe 10 of the present invention provides a separate design to enable the injection barrel 40 to be used with the needle 12 or the needle-free dosing connector 16 without the need for a new mold. This can reduce waste of material and lower manufacturing costs.

What is claimed is:

1. A safety syringe comprising;
a needle seat having a first shaft hole at a rear end thereof;
an adapter having a second shaft hole through front and rear ends thereof, the second shaft hole of the adapter being sleeved on the rear end of the needle seat and communicating with the first shaft hole of the needle seat, an outer surface of the adapter having an elastic wing;
an injection barrel having a barrel body and a shaft portion integrally connected with a front end of the barrel body and detachably penetrated into the first shaft hole of the needle seat and the second shaft hole of the adapter; and
an outer sleeve having a front opening and a rear opening, an inner surface of the outer sleeve having a first annular rib adjacent to the front opening and a second annular rib adjacent to the rear opening, the outer sleeve being sleeved on the injection barrel and movable along an axial direction of the injection barrel between a first position where the first annular rib of the outer sleeve is engaged with the elastic wing of the adapter, such that the outer sleeve is sleeved on the barrel body of the injection barrel and the needle seat is allowed to protrude from the front opening of the outer sleeve, and a second position where the outer sleeve accommodates therein the needle seat and the adapter and pushes against the elastic wind of the adapter through the second annular rib, such that the outer sleeve is detachable from the injection barrel together with the needle seat and the adapter.

2. The safety syringe as claimed in claim 1, wherein an outer surface of the needle seat has an outer flange, and an inner surface of the adapter has an inner flange interfered with the outer flange of the needle seat.

3. The safety syringe as claimed in claim 1, wherein an outer surface of the barrel body of the injection barrel has an elongated rib forced against the second annular rib of the outer sleeve when the outer sleeve is located at the first position.

4. The safety syringe as claimed in claim 1, wherein an inner surface of the outer sleeve has a third annular rib adjacent to the second annular rib and located between the first annular rib and the second annular rib; the elastic wing of the adapter is located between the second annular rib and the third annular rib when the outer sleeve is located at the second position.

5. The safety syringe as claimed in claim 4, wherein an inner diameter of the second annular rib is greater than an inner diameter of the first annular rib and an inner diameter of the third annular rib.
